(19) United States
(12) Patent Application Publication
(10) Pub. No.: US 2024/0350641 A1
(43) Pub. Date: Oct. 24, 2024

(54) DRUG CARRIER COMPOSITION AND HYPOGLYCEMIC DRUG

(71) Applicant: BEIJING RONGXIANG INSTITUTE OF REGENERATIVE MEDICINE CO., LTD., Beijing (CN)

(72) Inventor: Shijie SHEN, Beijing (CN)

(21) Appl. No.: 18/522,933

(22) Filed: Nov. 29, 2023

Related U.S. Application Data
(63) Continuation of application No. PCT/CN2023/121033, filed on Sep. 25, 2023.

(30) Foreign Application Priority Data
Apr. 23, 2023 (CN) 202310442156.4

(51) Int. Cl. A61K 47/36; A61K 31/353; A61K 31/4745; A61K 31/575; A61K 47/44

(52) U.S. Cl. CPC A61K 47/36; A61K 31/353; A61K 31/4745; A61K 31/575; A61K 47/44

(57) ABSTRACT

A drug carrier composition includes the following components in parts by weight: 10-25 parts of sesame oil, 2-8 parts of beeswax, and 0.6-1.4 parts of plant polysaccharide; the plant polysaccharide is one or more selected from the group consisting of aloe polysaccharide, Gardenia polysaccharide, and Trametes robiniophia murr polysaccharide.

### CROSS-REFERENCE TO RELATED APPLICATION

This application is a continuation of PCT application serial no. PCT/CN2023/121033, filed on Sep. 25, 2023, which claims the priority and benefit of Chinese patent application serial no. 202310442156.4, filed on Apr. 23, 2023. The entireties of PCT application serial no. PCT/CN2023/121033 and Chinese patent application serial no. 202310442156.4 are hereby incorporated by reference herein and made a part of this specification.

### TECHNICAL FIELD

The present application relates to a technical field of drug carrier, in particular, to a drug carrier composition and a hypoglycemic drug.

### BACKGROUND ART

Diabetes is an increasingly serious global epidemic, which is a metabolic disease group characterized by a chronic hyperglycemia. It is caused by various pathogenic factors, for example genetic factors, microbial infections, toxins, immune disorders, and mental factors, resulting in decreased pancreas islet function, insulin secretion defect, and impaired biological effect, or the both. The long-term hyperglycaemia can easily result in a chronic damage or a dysfunction of various tissues such as heart, blood vessel, kidney, eye, and nerve.

At present, the active ingredient in the diabetes drugs is prone to produce some side effects such as irritation and the like on the gastrointestinal tract, resulting in discomfort of the body. Therefore, a drug carrier composition that helps to reduce the irritation of diabetes drugs on the gastrointestinal tract as excipient is needed.

### SUMMARY

In order to reduce the irritation of diabetes drugs on the gastrointestinal tract, the present application provides a drug carrier composition and a hypoglycemic drug.

In a first aspect, the present application provides a drug carrier composition, including the following components in parts by weight: 10-25 parts of sesame oil, 2-8 parts of beeswax, and 0.6-1.4 parts of plant polysaccharide; the plant polysaccharide is one or more selected from the group consisting of aloe polysaccharide, Gardenia polysaccharide, and Trametes robiniophia murr polysaccharide.

In the present application, one or more of aloe polysaccharide, Gardenia polysaccharide, and Trametes robiniophia murr polysaccharide are used as plant polysaccharide, combined with sesame oil and beeswax to prepare a drug carrier composition. And when the drug carrier composition is used to prepare a hypoglycemic drug, the hypoglycemic drug has a good hypoglycemic effect, meanwhile the irritation of the drugs on the gastrointestinal tract is significantly reduced, and the discomfort symptoms of the body are alleviated.

In some embodiments, the drug carrier composition includes the following components in parts by weight: 18-25 parts of sesame oil, 2-5 parts of beeswax, and 0.6-1 parts of plant polysaccharide.

In some embodiments, the plant polysaccharide includes the aloe polysaccharide and the Gardenia polysaccharide.

In some embodiments, the plant polysaccharide includes the aloe polysaccharide and the Gardenia polysaccharide in a weight ratio of (0.5-1.5):(0.5-1).

In a specific embodiment, a weight ratio of the aloe polysaccharide and the Gardenia polysaccharide is 0.5:0.5, 0.5:1, or 1:0.5.

In a specific embodiment, a weight ratio of the aloe polysaccharide and the Gardenia polysaccharide is (0.5-1):0.5, (0.5-1):1, (1-1.5):0.5, or 1:(0.5-1).

Through experimental analysis, it can be known that combination of the aloe polysaccharide and the Gardenia polysaccharide in the above weight ratio is selected as plant polysaccharide in the present application, which can further improve the hypoglycemic effect of the hypoglycemic drugs, reduce the irritation of the drugs to the gastrointestinal tract, and greatly alleviate the discomfort symptoms of the body after taking the drugs.

In a second aspect, the present application provides a hypoglycemic drug, including the above drug carrier composition and an active ingredient.

In some embodiments, the active ingredient includes the following components in parts by weight: 2-5 parts of berberine hydrochloride, 0.5-1 parts of oryzanol, and 0.2-0.8 parts of tea polyphenol.

In some embodiments, a weight ratio of the active ingredient and the drug carrier composition is 1:(1-20).

In a specific embodiment, a weight ratio of the active ingredient and the drug carrier composition is 1:1, 1:5, 1:10, or 1:20.

In a specific embodiment, a weight ratio of the active ingredient and the drug carrier composition is 1:(1-5), 1:(1-10), 1:(5-10), 1:(5-20) or 1:(10-20).

Through experimental analysis, it can be known that the weight ratio of the active ingredient and the drug carrier composition is set in the range of the above weight ratio, which can further improve the hypoglycemic effect of the hypoglycemic drugs, and relieve the irritation of the drugs to the gastrointestinal tract.

The hypoglycemic drugs of the present application further includes a pharmaceutically acceptable adjuvant or diluent, besides above drug carrier composition and the active ingredient.

The hypoglycemic drugs of the present application may be formulated to be various dosage form known in the pharmaceutical field, such as tablet, granula, paste, suppository, cream, syrup, cream, ointments, gel, capsule, balsam, and plaster, according to specific administration ways.

The present application further provides a method for preparing the hypoglycemic drugs, including the following steps:
mixing the sesame oil, the beeswax and the plant polysaccharide in parts by weight, heating at 90-180° C., while sufficiently stirring, to obtain a drug carrier composition;
mixing the berberine hydrochloride, the oryzanol and the tea polyphenol, while sufficiently stirring, to obtain an active ingredient;
cooling the drug carrier composition to 60-90° C., at room temperature, then adding the active ingredient, and fully mixing the active ingredient with the drug carrier composition; and
vacuum degassing to obtain the hypoglycemic drugs.

In summary, the present application can achieve at least one of the following beneficial technical effects:
in the present application, one or more of aloe polysaccharide, Gardenia polysaccharide, and Trametes robiniophia murr polysaccharide are used as plant polysaccharide, combined with sesame oil and beeswax as drug carrier composition, and further the hypoglycemic drug is prepared. The prepared hypoglycemic drug not only has excellent anti diabetes effect, but also can overcome the adverse reactions such as gastrointestinal irritation caused by the active ingredient; and
in the present application, the type and usage amount of the plant polysaccharide are further studied, meanwhile the active ingredient is also studied, so that the hypoglycemic effect of the hypoglycemic drug is further improved, the irritation of the drugs to the gastrointestinal tract is reduced, and the discomfort symptoms generated in the body after taking the drugs are greatly alleviated.

### BRIEF DESCRIPTION OF THE DRAWINGS

FIG. 1 is the detection results of fasting blood glucose before and after administrating the drugs in Example 2, Comparative example 1, Comparative example 3, the model group and the blank control group.

### DETAILED DESCRIPTION

In a first aspect, the present application provides a drug carrier composition, including the following components in parts by weight: 10-25 parts of sesame oil, 2-8 parts of beeswax, and 0.6-1.4 parts of plant polysaccharide; the plant polysaccharide is one or more selected from the group consisting of aloe polysaccharide, Gardenia polysaccharide, and Trametes robiniophia murr polysaccharide.

Specifically, the plant polysaccharide includes the aloe polysaccharide and the Gardenia polysaccharide in a weight ratio of (0.5-1.5):(0.5-1).

In a second aspect, the present application provides a hypoglycemic drug, including the above drug carrier composition and an active ingredient.

The active ingredient includes the following components in parts by weight: 2-5 parts of berberine hydrochloride, 0.5-1 parts of oryzanol, and 0.2-0.8 parts of tea polyphenol; a weight ratio of the active ingredient and the drug carrier composition is 1:(1-20).

The present application provides a method for preparing the hypoglycemic drugs, including the following steps:
mixing the sesame oil, the beeswax and the plant polysaccharide in parts by weight, heating at 90-180° C., while sufficiently stirring, to obtain a drug carrier composition;
mixing the berberine hydrochloride, the oryzanol and the tea polyphenol, while sufficiently stirring, to obtain an active ingredient;
cooling the drug carrier composition to 60-90° C., at room temperature, then adding the active ingredient, and fully mixing the active ingredient with the drug carrier composition; and
vacuum degassing to obtain the hypoglycemic drugs.

The present application will be further described in detail with reference to the examples, the comparative examples and the performance test. These examples cannot be anticipated as a limit to the protection scope of the present application.

In the present application, the aloe polysaccharide, the Gardenia polysaccharide, and the Trametes robiniophia murr polysaccharide used in the examples and the lentinan used in the comparative examples are all available from Shanghai Yuanye Biotechnology Co., Ltd; and other raw materials can be commercially available.

### EXAMPLE

### Examples 1-11

Examples 1-11 provide a hypoglycemic drug respectively.

Differences between the above examples are addition amounts of each component in the drug carrier composition of the hypoglycemic drugs, as shown in Table 1.

The method for preparing the hypoglycemic drugs in the above examples, includes the following steps:
the sesame oil, the beeswax and the plant polysaccharide were weighted according to Table 1, respectively, and mixed, then heated at 150° C., while sufficiently stirring for 15 minutes to obtain a drug carrier composition;
3.5 g of berberine hydrochloride and 0.8 g of oryzanol were weighted, added into a vertical stirring ball mill, mechanically activated at a speed of 500 r/min for 3 minutes, 0.5 g of the tea polyphenols was added, stirred and mixed, to obtain 4.8 g of the active ingredients;
24 g of the drug carrier composition was weighted and cooled to 75° C., at room temperature, then the active ingredients were added, and stirred for 15 minutes, so that the active ingredients and the drug carrier composition were fully mixed; in particular, the weight ratio of the active ingredients and the drug carrier composition was 1:5;
a vacuum degassing was performed to obtain the hypoglycemic drugs.

TABLE 1
The usage amount of each component in the drug carrier composition of Examples 1-11

| | | | Examples | | | | | | | | | | |
|---|---|---|---|---|---|---|---|---|---|---|---|---|---|
| | | | 1 | 2 | 3 | 4 | 5 | 6 | 7 | 8 | 9 | 10 | 11 |
| Usage amount | Sesame oil | | 18 | 18 | 18 | 18 | 18 | 18 | 18 | 18 | 18 | 10 | 25 |
| of each | Beeswax | | 5 | 5 | 5 | 5 | 5 | 5 | 5 | 5 | 5 | 8 | 2 |
| component | Plant | Aloe | 0.5 | 1 | 1.5 | 2 | 1 | — | — | 1 | — | 1 | 1 |
| in the drug | polysaccharide | polysaccharide | | | | | | | | | | | |
| carrier | | Gardenia | 1 | 0.5 | 0.5 | 0.5 | — | 1 | 1 | — | — | 0.5 | 0.5 |
| composition/g | | polysaccharide | | | | | | | | | | | |
| | | Trametes | — | — | — | — | — | — | 0.5 | 0.5 | 1 | — | — |
| | | robiniophia | | | | | | | | | | | |
| | | murr | | | | | | | | | | | |
| | | polysaccharide | | | | | | | | | | | |

| | | | | | | | | | | | |
|---|---|---|---|---|---|---|---|---|---|---|---|
| Usage amount of plant | 1 | 1 | 1 | 1 | 1 | 1 | 1 | 1 | 1 | 1.4 | 0.6 |
| polysaccharide/g | | | | | | | | | | | |

| | | | | | | | | | | | |
|---|---|---|---|---|---|---|---|---|---|---|---|
| Usage amount of the drug carrier composition/g | 24 | 24 | 24 | 24 | 24 | 24 | 24 | 24 | 24 | 24 | 24 |

“—” refers to not added

### Examples 12-17

Examples 12-17 provides a hypoglycemic drug, respectively.

In particular, the hypoglycemic drugs of Examples 12-13 were same as that of Example 2, except that the addition amount of each component in the active ingredient were different.

The hypoglycemic drugs of Examples 14-17 were same as that of Example 2, except that the weight ratio of the active ingredient to the drug carrier composition were different, as shown in Table 2.

TABLE 2
Addition amount of each component in the active ingredient,
and weight ratio of the active ingredient to the drug
carrier composition in Example 2 and Examples 12-17

| | | Example | | | | | | |
|---|---|---|---|---|---|---|---|---|
| | | 2 | 12 | 13 | 14 | 15 | 16 | 17 |
| Usage amount of | Berberine | 3.5 | 2 | 5 | 3.5 | 3.5 | 3.5 | 3.5 |
| each component | hydrochloride | | | | | | | |
| in the active | Oryzanol | 0.8 | 0.5 | 1 | 0.8 | 0.8 | 0.8 | 0.8 |
| ingredient/g | Tea polyphenol | 0.5 | 0.2 | 0.8 | 0.5 | 0.5 | 0.5 | 0.5 |

| | | | | | | | |
|---|---|---|---|---|---|---|---|
| Usage amount of the active ingredient/g | 4.8 | 4.8 | 4.8 | 4.8 | 1.2 | 4.8 | 1.2 |
| Usage amount of the drug carrier | 24 | 24 | 24 | 4.8 | 24 | 2.4 | 30 |
| composition/g | | | | | | | |
| Weight ratio of the active ingredient | 1:5 | 1:5 | 1:5 | 1:1 | 1:20 | 1:0.5 | 1:25 |
| to the drug carrier composition | | | | | | | |

### COMPARATIVE EXAMPLE

### Comparative Example 1

The present comparative example provides a hypoglycemic drug.

The hypoglycemic drug of this comparative example was same as that of example 2, except that the plant polysaccharide was not added to the drug carrier composition in this comparative example; that is, the drug carrier composition was made of the sesame oil and beeswax in a weight ratio of 18:5.

### Comparative Example 2

The present comparative example provides a hypoglycemic drug.

The hypoglycemic drug of this comparative example was same as that of example 2, except that the plant polysaccharide was 1 g of the lentinan in the drug carrier composition in this comparative example.

### Comparative Example 3

The present comparative example provides a hypoglycemic drug.

The hypoglycemic drug of this comparative example was same as that of example 2, except that the tea polyphenol was not added to the active ingredient in this comparative example; that is, the active ingredient was made of the berberine hydrochloride and oryzanol in a weight ratio of 3.5:0.8.

### Performance Tests:

The hypoglycemic drugs prepared by Examples 1-17 and Comparative examples 1-3 were tested for the application effect, using mouse animal experiments.

The test method was as follows:
the hypoglycemic drugs prepared by Examples 1-17 and Comparative examples 1-3 were dispersed into the edible vegetable oil, to formulate a hypoglycemic drug solution with a concentration of 20 mg/mL.

Establishing diabetes mouse model:C57BL/6 mice aged 6 weeks of SPF grade (18-23 g of weight) were adaptively raised for 7 days, and then divided into two groups, one group of 5 was fed with basic diet, as a blank control group; the other group of 200 was fed with high fat and high sugar diet; after 3 weeks, the mouse were fasted but given water for 12 h; and the fasting blood glucose was measured by collecting vein blood from the tail of each mouse. The mice having a blood glucose of greater than 12 mmol/L were selected as diabetes model mouse for subsequent tests.

Administration treatment: diabetes model mice were randomly divided into 22 groups, each group was 5; in particular, one group was Model Group, in which each mouse was gavaged with 2 mL of the edible vegetable oil every day for 3 weeks; and the remaining 21 groups of diabetes model mice were Experimental Groups, in which each mouse was gavaged with 2 mL of the hypoglycemic drug solution of the examples 1-17 and the comparative examples 1-3, respectively, every day for 3 weeks. In the experimental process, the untreated group was blank control group: each mouse was gavaged with 2 mL of the edible vegetable oil every day for 3 weeks.

### Test:

(1) Observation of discomfort symptoms: after the administration treatment, the mice were weighted every other week (the 7th, 14th, and 21st days), and conditions of the mice were observed for any discomfort symptoms such as vomit, diarrhea, body torsion and the like, and scored according to the scoring criteria given in Table 3; And the weight indicator, vomit indicator, diarrhea indicator, and body torsion indicator were indicated with labels A, B, C, and D.

TABLE 3
Scoring criteria for discomfort symptoms of the mice

| Indicator | Scoring Criteria | score | Indicator | Scoring Criteria | score |
|---|---|---|---|---|---|
| Weight | NO Declined | 0 | Vomit | NO | 0 |
| (A) | Decrease by 1% ≤ A < 6% | 5 | (B) | A small amount of vomit | 5 |
| | Decrease by 6% ≤ A < 12% | 10 | | A small amount of vomit | 10 |
| | | | | with retching | |
| | Decrease by 12% ≤ A < 18% | 15 | | A large amount of vomit | 15 |
| | | | | with retching | |
| Diarrhea | Normal feces | 0 | Body | 0 < D ≤ 5 times/30 min | 0 |
| (C) | Loose feces | 5 | torsion | 5 < D ≤ 10 times/30 min | 5 |
| | Mild diarrhea in feces | 10 | (D) | 10 < D ≤ 20 times/30 min | 10 |
| | Severe diarrhea in feces | 15 | | 20 < D ≤ 30 times/30 min | 15 |

Detection results: After the drugs of Examples 1-17 and Comparative examples 1-3 were administrated to the experimental mice, the test results of discomfort symptoms were shown in Table 4.

TABLE 4
The detection results of discomfort symptoms caused after administrating
the hypoglycemic drugs of Examples 1-17 and Comparative examples 1-3.

| | | Test results of discomfort symptoms | | | | | | | | | | | | |
|---|---|---|---|---|---|---|---|---|---|---|---|---|---|---|
| | | The 7th day | | | | The 14th day | | | | The 21st day | | | | Total |
| Test object | | A | B | C | D | A | B | C | D | A | B | C | D | scores |
| Examples | 1 | 0 | 0 | 0 | 0 | 0 | 0 | 0 | 0 | 0 | 0 | 0 | 0 | 0 |
| | 2 | 0 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 | 0 |
| | 4 | 0 | 0 | 5 | 0 | 0 | 0 | 0 | 5 | 0 | 0 | 0 | 0 | 10 |
| | 5 | 0 | 5 | 0 | 5 | 0 | 0 | 0 | 0 | 0 | 5 | 0 | 0 | 15 |
| | 6 | 0 | 0 | 5 | 0 | 0 | 5 | 0 | 5 | 0 | 0 | 0 | 0 | 15 |
| | 7 | 0 | 5 | 5 | 0 | 0 | 5 | 5 | 5 | 0 | 5 | 5 | 5 | 40 |
| | 8 | 0 | 5 | 0 | 0 | 0 | 0 | 5 | 5 | 5 | 10 | 5 | 0 | 35 |
| | 9 | 0 | 5 | 5 | 0 | 0 | 5 | 0 | 5 | 5 | 5 | 5 | 5 | 40 |
| | 10 | 0 | 0 | 0 | 0 | 0 | 0 | 0 | 0 | 0 | 0 | 0 | 0 | 0 |
| | 11 | 0 | 5 | 0 | 0 | 0 | 0 | 0 | 0 | 0 | 0 | 0 | 0 | 5 |
| | 12 | 0 | 0 | 5 | 0 | 0 | 0 | 0 | 0 | 0 | 0 | 0 | 0 | 5 |
| | 13 | 0 | 0 | 0 | 0 | 0 | 0 | 0 | 0 | 0 | 0 | 0 | 0 | 0 |
| | 14 | 0 | 0 | 0 | 0 | 0 | 5 | 0 | 0 | 0 | 0 | 0 | 0 | 5 |
| | 15 | 0 | 5 | 0 | 0 | 0 | 0 | 0 | 0 | 0 | 0 | 0 | 0 | 5 |
| | 16 | 0 | 0 | 5 | 0 | 0 | 5 | 0 | 0 | 0 | 0 | 0 | 0 | 10 |
| | 17 | 0 | 0 | 0 | 0 | 0 | 0 | 0 | 0 | 0 | 0 | 0 | 0 | 0 |
| Comparative | 1 | 0 | 15 | 10 | 15 | 5 | 15 | 10 | 5 | 15 | 15 | 15 | 10 | 130 |
| examples | 2 | 0 | 10 | 15 | 10 | 5 | 10 | 15 | 0 | 10 | 15 | 10 | 5 | 105 |
| | 3 | 0 | 10 | 10 | 10 | 5 | 10 | 10 | 5 | 15 | 15 | 15 | 10 | 115 |

| | | | | | | | | | | | | | |
|---|---|---|---|---|---|---|---|---|---|---|---|---|---|
| Model group | 5 | 0 | 5 | 5 | 0 | 5 | 10 | 10 | 0 | 10 | 10 | 10 | 70 |
| Blank control | 0 | 0 | 0 | 0 | 0 | 0 | 0 | 0 | 0 | 0 | 0 | 0 | 0 |
| group | | | | | | | | | | | | | |

(2) Detection of bacterial colony in the feces: after administration treatment, that is, the 21st day, the microorganism DNA was extracted from the mouse feces according to instructions of DNA extraction kit of intestinal contents (available from Tiangen Biochemical Technology Co., Ltd). Total DNA was measured by Thermo NanoDrop 2000 UV micro spectrophotometer and 1% agar gel electrophoresis. The diluted genomes DNA was used as a template, and KAPA HiFi Hotstart ReadyMix PCR kit high-fidelity enzyme was used for PCR, which ensures the accuracy and efficiency of the amplification. The PCR products were measured by 2% agarose gel electrophoresis and recovered by AxyPrep DNA gel recovery kit. After the recovery, the library was checked by Thermo NanoDrop 2000 UV micro spectrophotometer and 2% agarose gel electrophoresis. After the library passes the quality inspection, the library was quantified by Qubit, and the corresponding proportion was mixed according to the data volume requirements of each sample. The V3-V4 regions of 16s rDNA in the intestinal flora was sorted and sequenced. The sequencing results were analyzed for bioinformatics.

TABLE 5
The detection results of intestinal floras after administrating the
hypoglycemic drugs of Examples 1-17 and Comparative examples 1-3

| | | Detection results | | |
|---|---|---|---|---|
| | | Abundance of beneficial bacteria | | |
| | | producing the short-chain fatty acids | | |
| Detection object | | Lachnospira | Prevotella | Ruminococcus |
| Example | 1 | 3.15 | 1.26 | 6.43 |
| | 2 | 3.23 | 1.28 | 6.58 |
| | 3 | 3.12 | 1.32 | 6.39 |
| | 4 | 2.92 | 1.15 | 6.23 |
| | 5 | 2.87 | 1.18 | 6.19 |
| | 6 | 2.89 | 1.14 | 6.21 |
| | 7 | 2.65 | 1.01 | 6.01 |
| | 8 | 2.71 | 1.09 | 5.98 |
| | 9 | 2.62 | 0.95 | 5.84 |
| | 10 | 3.19 | 1.29 | 6.48 |
| | 11 | 3.01 | 1.19 | 6.25 |
| | 12 | 3.05 | 1.18 | 6.27 |
| | 13 | 3.14 | 1.30 | 6.53 |
| | 14 | 2.91 | 1.14 | 6.25 |
| | 15 | 2.98 | 1.16 | 6.13 |
| | 16 | 2.94 | 1.14 | 6.21 |
| | 17 | 3.20 | 1.25 | 6.48 |
| Comparative | 1 | 0.15 | 0.57 | 2.23 |
| example | 2 | 0.18 | 0.56 | 3.09 |
| | 3 | 0.85 | 0.86 | 4.56 |

| | | | |
|---|---|---|---|
| Model group | 1.13 | 0.69 | 3.06 |
| Blank control group | 3.38 | 1.51 | 6.62 |

According to the detection results of Table 5, it can be seen that, administration of the hypoglycemic drugs of the present application to the diabetes model mice can adjust the dynamic balance of the intestinal floras, increase the abundance of the beneficial bacteria in the intestinal tract, reduce the abundance of harmful bacteria, and increases the generation of the short-chain fatty acids in the intestinal tract.

(3) Fasting blood glucose: After the administration treatment, every other week (the 7th, 14th, and 21st days), all the mice were in the fasted but given water for 4 h, and the fasting blood glucose was measured by collecting vein blood from the tail of each mouse.

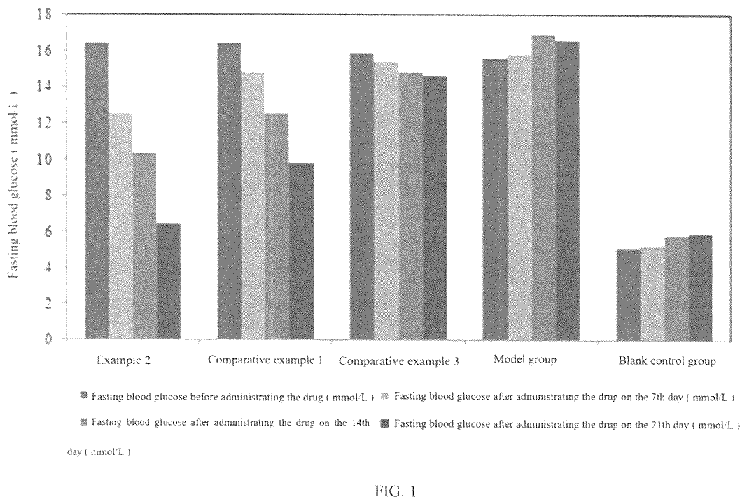

Test results: FIG. 1 was the detection results of fasting blood glucose before and after administrating the drugs in Example 2, Comparative example 1, Comparative example 3, the model group and the blank control group.

The detection results of hypoglycemic drugs of Examples 1-17 and Comparative examples 1-3 were shown in Table 6.

TABLE 6
The detection results of hypoglycemic drugs of
Examples 1-17 and Comparative examples 1-3.

| | | Detection results | | | |
|---|---|---|---|---|---|
| | | Fasting blood glucose (mmol/L) | | | |
| | | Before | | | |
| | | administrating | After administrating drugs | | |
| Detection object | | drugs | 7^{th} day | 14^{th} day | 21^{st} day |
| Examples | 1 | 16.2 | 13.8 | 11.2 | 7.2 |
| | 2 | 16.4 | 12.5 | 10.3 | 6.4 |
| | 3 | 15.8 | 13.2 | 10.8 | 6.9 |
| | 4 | 16.1 | 14.3 | 12.5 | 8.8 |
| | 5 | 16.0 | 14.9 | 12.3 | 10.2 |
| | 6 | 16.2 | 15.3 | 12.8 | 10.5 |
| | 7 | 15.7 | 14.1 | 11.8 | 8.9 |
| | 8 | 16.5 | 14.3 | 11.6 | 9.2 |
| | 9 | 15.7 | 15.1 | 13.8 | 11.2 |
| | 10 | 15.9 | 13.6 | 11.5 | 8.8 |
| | 11 | 15.8 | 12.8 | 10.8 | 7.9 |
| | 12 | 16.5 | 14.2 | 11.8 | 8.2 |
| | 13 | 15.8 | 13.3 | 10.7 | 7.8 |
| | 14 | 16.3 | 12.5 | 10.5 | 7.2 |
| | 15 | 15.9 | 13.2 | 11.3 | 8.9 |
| | 16 | 16.1 | 12.6 | 10.5 | 6.8 |
| | 17 | 16.2 | 14.2 | 12.8 | 9.9 |
| Comparative | 1 | 16.4 | 14.8 | 12.5 | 9.8 |
| examples | 2 | 16.2 | 14.9 | 11.8 | 9.2 |
| | 3 | 15.9 | 15.4 | 14.8 | 14.6 |

| | | | | |
|---|---|---|---|---|
| Model Group | 15.6 | 15.8 | 16.9 | 16.6 |
| Blank control group | 5.1 | 5.2 | 5.8 | 5.9 |

Referring to Table 4, Table 5, Table 6 and comparing the detection results of Examples 1-17, Comparative examples 1-3, the model group and the blank control group, it can be seen that, in the present application, one or more of the aloe polysaccharide, the Gardenia polysaccharide, the Trametes robiniophia murr polysaccharide were used as the plant polysaccharide, the sesame oil and the beeswax were used as the drug carrier composition, combined with the active ingredient to prepare the hypoglycemic drugs. After the diabetes mouse model was administrated the drugs for 3 weeks, the fasting blood glucose of the diabetes mouse model was decreased to 6.4-11.2 mmol/L from 15.7-16.4 mmol/L. Moreover, the discomfort symptoms of the diabetes mouse model were less, and score of the discomfort symptoms was 0-40. The detection results indicated when the drug carrier composition of the present application was used in the hypoglycemic drugs, the irritation of the hypoglycemic drug to the gastrointestinal tract can be reduced on the basis of ensuring the hypoglycemic effect when preparing hypoglycemic drugs.

Comparing the detection results of Example 1 and Comparative examples 1-2, it can be seen that, one or more of the aloe polysaccharide, the Gardenia polysaccharide, the Trametes robiniophia murr polysaccharide were used as plant polysaccharide in the present application, such that the irritation of drugs to the gastrointestinal tract was reduced, compared to no addition of the plant polysaccharide or addition of the lentinan polysaccharide as plant polysaccharide.

Comparing the detection results of Examples 1-9, it can be seen that, compared to that the Trametes robiniophia murr polysaccharide alone was selected as the plant polysaccharide, or the Trametes robiniophia murr polysaccharide and the aloe polysaccharide were selected as the plant polysaccharide, or the Trametes robiniophia murr polysaccharide and the Gardenia polysaccharide were selected as the plant polysaccharide, one or more of the aloe polysaccharide and the Gardenia polysaccharide were used as the plant polysaccharide in the present application can improve the hypoglycemic effect of the drugs and reduce the irritation of drugs to the gastrointestinal tract. Preferably, the aloe polysaccharide and the Gardenia polysaccharide in a weight ratio of (0.5-1.5):(0.5-1) were selected as the plant polysaccharide.

Comparing the test results of Example 2 and Comparative example 3, it can be seen that, the tea polyphenol was selected as the active ingredient of the hypoglycemic drug in the present application, which can significantly improve the hypoglycemic effect of the drug and reduce the irritation of the drugs to the gastrointestinal tract, compared to no addition of the active ingredient of tea polyphenol.

Comparing the test results of Example 2 and Examples 14-17, it can be seen that, when the weight ratio of the active ingredient and the drug carrier composition was greater than 1:1, the drug had a certain irritation to the gastrointestinal tract; when the weight ratio of the active ingredient and the drug carrier composition was less than 1:20, the hypoglycemic effect of the drug was reduced. Therefore, the weight ratio of the active ingredient and drug carrier composition was controlled to be 1:(1-20), which can ensure a good hypoglycemic effect, while reducing the irritation of the drugs to the gastrointestinal tract.

Although the present application has been described in detail with general explanations and specific embodiments above, it is obvious to those skilled in the art that some modifications or improvements can be made based on the present invention. Therefore, these modifications or improvements made based on the spirit of the present application fall within the protection scope of the present application.

What is claimed is:

1. A drug carrier composition, comprising the following components in parts by weight: 10-25 parts of sesame oil, 2-8 parts of beeswax, and 0.6-1.4 parts of plant polysaccharide; wherein the plant polysaccharide is one or more selected from the group consisting of aloe polysaccharide, Gardenia polysaccharide, and Trametes robiniophia murr polysaccharide.

2. The drug carrier composition according to claim 1, wherein the drug carrier composition comprises the following components in parts by weight: 18-25 parts of the sesame oil, 2-5 parts of the beeswax, and 0.6-1 parts of the plant polysaccharide.

3. The drug carrier composition according to claim 1, wherein the plant polysaccharide comprises the aloe polysaccharide and the Gardenia polysaccharide.

4. The drug carrier composition according to claim 1, wherein the plant polysaccharide comprises the aloe polysaccharide and the Gardenia polysaccharide in a weight ratio of (0.5-1.5):(0.5-1).

5. A hypoglycemic drug, comprising the drug carrier composition of claim 1 and an active ingredient.

6. The hypoglycemic drug according to claim 5, wherein the active ingredient comprises the following components in parts by weight: 2-5 parts of berberine hydrochloride, 0.5-1 parts of oryzanol, and 0.2-0.8 parts of tea polyphenol.

7. The hypoglycemic drug according to claim 5, wherein a weight ratio of the active ingredient and the drug carrier composition is 1:(1-20).

8. The hypoglycemic drug according to claim 5, wherein a dosage form of the hypoglycemic drug is any one selected from the group consisting of tablet, granula, paste, suppository, cream, syrup, cream, ointments, gel, capsule, balsam, and plaster.
